(19) United States
(12) Patent Application Publication
(10) Pub. No.: US 2004/0213757 A1
(43) Pub. Date: Oct. 28, 2004

(54) Water soluble wortmannin derivatives

(75) Inventors: Tianmin Zhu, Monroe, NY (US); Ker Yu, Pine Brook, NJ (US); Judy Lucas, Nanuet, NY (US); Jianxin Gu, River Edge, NJ (US); Arie Zask, New York, NY (US)

Correspondence Address: WYETH, PATENT LAW GROUP, 5 GIRALDA FARMS, MADISON, NJ, 07940, US

(73) Assignee: Wyeth Holdings Corporation, Madison, NJ

(21) Appl. No.: 10/828,474

(22) Filed: Apr. 20, 2004

Related U.S. Application Data
(60) Provisional application No. 60/464,796, filed on Apr. 23, 2003.

(51) Int. Cl. A61K 31/765; C08L 37/00

(52) U.S. Cl. 424/78.19; 525/206

(57) ABSTRACT

This invention relates to soluble derivatives of wortmannin that utilizes water-soluble polymers as carriers for a drug and includes compounds having the structures as described within the specification.

This application claims priority from copending provisional application Ser. No. 60/464,796, filed Apr. 23, 2003, the entire disclosure of which is hereby incorporated by reference.

### BACKGROUND OF THE INVENTION

Wortmannin is a fungal metabolite found to be a potent catalytic inhibitor of phosphatidylinositol-3(OH)-kinase (PI3K) and TOR kinase function within signal transduction pathways. (Norman, Bryan H., et al. (1996) “Studies on the Mechanism of the Phosphatidylinositol 3-Kinase Inhibition by Wortmannin and Related Analogs”, J. Med. Chem., 39, 1106-111 and Creemer, Lawrence C. (1996) “Synthesis and in Vitro Evaluation of New Wortmannin Esters: Potent Inhibitors of Phosphatidylinositol 3-Kinase”, J. Med. Chem, 39, 5021-5024).

The class-1a PI3K (referred to as PI3K) is a heterodimeric enzyme comprised of the p85 regulatory and p110 catalytic subunits. In response to growth factor receptor stimulation, PI3K catalyzes the production of the lipid second messenger phosphatidylinositol-3,4,5-triphosphate (PIP3) at the cell membrane. PIP3 in turn contributes to the activation of a wide range of downstream cellular substrates. The most critical signaling mediators downstream of PI3K include the serine/threonine kinase AKT and the mammalian target of rapamycin (mTOR). AKT confers a dominant survival signal and promotes proliferation via direct phosphorylation of multiple cell death/apoptosis proteins and cell cycle factors. mTOR is a central regulator of cell growth via controlling of cellular protein translation. Thus, the PI3K/AKT/TOR pathway is critical for cell proliferation, growth, survival and angiogenesis. In human cancer, deregulation in the PI3K/AKT/TOR pathway is among the most frequent events occurring in all major human tumors. Genetic loss of the tumor suppressor gene PTEN, a PIP3 phosphatase and a negative regulator of the PI3K signaling, is estimated to occur in 30-50% of all human cancers including lung, prostate, breast, brain, renal, melanoma, ovarian, endometrium, thyroid and lymphoid. In addition, constitutive elevation of PI3K expression has been associated with lung, ovarian and pancreatic cancers. Finally, cell surface oncogenes such as Her-2, EGFR and Ras cause constitutive PI3K signaling in breast, prostate, colon and lung tumors. These clinical data provide a strong rationale for exploring PI3K inhibitors as novel anticancer agents. (Cantley, L. and Neel, B. (1999) “New Insights into Tumor Suppression: PTEN Suppresses Tumor Formation by Restraining the Phosphoinositide 3-kinase/AKT pathway”, Proc. Natl. Acad. Sci. USA, 96, 4240-4245). PI 3 kinase and TOR kinase have been shown to be active in cancer (Vivanco, I. and Sawyer, C. (2002) “The phosphatidylinositol 3-kinase-AKT Pathway in Human Cancer”, Nature Reviews Cancer, 2, 489-501), iscaemic heart disease and restenosis (Shiojima, I. And Walsh, K. (2002) “Role of Akt Signaling in Vascular Homeostasis and Angiogenesis”, Circulation Research, 90, 1243-1250 and Ruygrok P., et al. (2003) “Rapamycin in Cardiovascular Medicine”, Intern Med J., 33, 103-109), inflammation (Wymann, M., et al., (2003) “Phosphoinostide 3-kinase gamma: A Key Modulator in Inflammation and Allergy” Biochem Soc Trans, 31, 275-280 and Kwak, Yong-Geun, et al., (April 2003) “Involvement of PTEN in airway hyperresponsiveness and inflammation in bronchial asthma”, The Journal of Clinical Investigation, 111:7, 1083-1092), platelet aggregation (Watanabe, N., et al. (March 2003) “Functional Phenotype of Phosphoinositide 3-kinase p85 (alpha) Null Platelets Characterized by an Impaired Response to GP VI Stimulation”, Blood (epub)), sclerosis (Kenerson, H., et al. (2002) “Activated Mammalian Target of Rapamycin in the Pathogenesis of Tuberous Sclerosis Complex Renal Tumors”, Cancer Res., 62, 5645-5650), respiratory disorders (Kitaura, J., et al., (2000) “AKT-dependent Cytokine Production in Mast Cells”, J. Exp. Med., 192, 729-739 and Stewart A. (2001) “Airway Wall Remodeling and Hyper-responsiveness: Modeling Remodeling in vitro and in vivo”, Pulm Pharmacol Ther, 14, 255-265), HIV (Francois, F. and Klotman, M. “Phosphatidylinositol 3-kinase Regulates Human Immunodeficiency Virus Type-1 Replication Following Viral Entry in Primary CD4(+) T Lymphocytes and Macrophages”, J. Virol., 77, 2539-2549), and bone resorption (Pilkington, M., et al. (1998) “Wortmannin Inhibits Spreading and Chemotaxis of Rat Osteoclasts in vitro”, J Bone Miner Res, 13, 688-694).

PI 3-kinase exists as a tightly associated heterodimer of an 85 kDa regulatory subunit and 110 kDa catalytic subunit, and is found in cellular complexes with almost all ligand-activated growth factor receptors and oncogene protein tyrosine kinases (Cantley, L. C., et al., Cell, 64:281-302 (1991)). The 85 kDa regulatory subunit apparently acts as an adaptor of PI 3-kinase to interact with growth factor receptors and tyrosine phosphorylated proteins (Margolis, C. Cell Growth Differ., 3:73-80 (1992)).

Although PI 3-kinase appears to be an important enzyme in signal transduction, with particular implications relative to mitogenesis and malignant transformation of cells, only a limited number of water-soluble drug-polymer conjugates have been identified as having inhibitory activity against PI 3-kinase (see, e.g., Matter, W. F., et al., Biochem. Biophys, Res. Commun., 186:624-631 (1992)). Contrary to the selective PI 3-kinase activity of the water-soluble drug-polymer conjugates used in the methods of the present invention, the bioflavinoid water-soluble drug-polymer conjugates used by Matter, et al., particularly quercetin and certain analogs thereof, inhibit PI 3-kinase and other kinases such as protein kinase C and PI 4-kinase (Id.).

U.S. Pat. No. 5,378,725, issued Jan. 3, 1995, provided a method for inhibiting PI 3-kinase in mammals using wortmannin or one of certain analogs thereof. One of the disadvantages of wortmannin is its toxicity to living creatures. Even in low dosages, wortmannin in pure form is often systemically dose limiting to laboratory animals.

The biosynthetic production of wortmannin is well known in the art and the derivatives are synthesized from wortmannin. (Dewald, Beatrice, et al. (1988) “Two Transduction Sequences Are Necessary for Neutrophil Activation by Receptor Agonists”, The Journal of Biological Chemistry, Vol. 263, Issue of November 5, pp 16179-16184; Norman, Bryan H., et al., (1996) “Studies on the Mechanism of Phosphatidylinositol 3-Kinase Inhibition by Wortmannin and Related Analogs”, J. Med. Chem., 39, pp 1106-1111; Varticovski, L., et a. (2001) “Water-soluble HPMA copolymer-wortmannin conjugate retains phosphoinositide 3-kinase inhibitory activity in vitro and in vivo”, Journal of Controlled Release, 74, pp 275-281), all hereby incorporated by reference.

A wortmannin derivative, 17β-Hydroxywortmannin prepared from the reduction of wortmannin with diborane, showed a 10-fold increase in activity relative to wortmannin and pushed the PI3K IC₅₀ into the subnanomolar range, with an IC₅₀ of 0.50 nM. However, antitumor activity of 17β-Hydroxywortmannin in the C3H mammary model showed no inhibition at a dose of 0.5 (mg/kg) and toxicity at a dose of 1.0 mg/kg. These findings lead the authors to conclude, “nucleophilic addition to the electrophilic C-21 position of wortmannin and related analogs is required for inhibitor potency and antitumor activity. Unfortunately, this mechanism appears to be linked to the observed toxicity” (Norman, Bryan H., et al. (1996) “Studies on the Mechanism of Phosphatidylinositol 3-Kinase Inhibition by Wortmannin and Related Analogs”, J. Med. Chem., 39, 1106-1111,1109-1110).

Wortmannin derivatives acetylated at the C-17 hydroxyl group showed a dramatic loss in activity leading the authors to conclude, “the active site cannot accommodate liphophilicity or steric bulk at C-17” (Creemer, Lawrence C., et al. (1996) “Synthesis and in Vitro Evaluation of New Wortmannin Esters: Potent Inhibitors of Phosphatidylinositol 3-Kinase”, J. Med. Chem., 39, 5021-5024, 5022). This conclusion is consistant with the X-ray crystallographic structure of PI3K bound to wortmannin subsequently elucidated (Walker, Edward H., et. al (2000) “Structural Determinants of Phosphoinositide 3-Kinase Inhibition by Wortmannin, LY294002, Quercetin, Myricetin, and Staurosporine”, Molecular Cell 6(4), 909-919).

Attaching poly(ethyleneglycol) (PEG) has been successfully employed in medicinal chemistry to improve the aqueous solubility and administration of drugs. (Id.) However, covalently attaching PEG does not necessarily offer improvement in water solubility and availability of the drug to which it is attached (Bebbington, David, et al. (2002) “Prodrug and Covalent Linker Strategies for the Solubilization of Dual-Action Antioxidants/Iron Chelators”, Bioorganic & Medicinal Chemistry Letters, 12, 3297-3300, 3299) and (Feng, Xia, et al. (2002) “Synthesis and Evaluation of Water-Soluble Paclitaxel Prodrugs”, Bioorganic & Medicinal Chemistry Letters, 12, 3301-3303, 3302).

In an overview of PEG drugs, no low molecular weight (<20,000) PEG small molecule drug conjugates, prepared over a 20-year period, have led to a clinically approved product (Greenwald, R. B. (2001) “PEG drugs: an overview”, Journal of Controlled Release, 74, pp 159-171, abstract). In fact only a few small organic molecule anticancer agents have been conjugated to PEG with permanent bonds, and those did not lead to clinically superior water-soluble drug-polymer conjugates (Greenwald, R. B., et al. (2003) “Effective Drug Delivery by PEGylated Drug Conjugates”, Advanced Drug Delivery Reviews, 55, pp 217-250, 220). Using PEG-CPT, lethality was demonstrated to be approximately 50%, 10% and 0% for the PEG-CPT 40,000, 20,000 and 8,000 constructs. Ostensibly, employing polymer M_{w} 5000 to conjugate drugs gave rapidly excreted species that would have little or no effect in vivo (Id., 225). That is not to say the attachment of PEG 40,000 with its ability to accumulate in tumors will automatically permit drugs to have greater antitumor activity (Id., 235).

In the present invention, to deliver wortmannin derivatives in a water-soluble form a water-soluble polymer was bound to the wortmannin derivative, the resultant polymer-drug conjugate being soluble. Binding water soluble polymers such as PEG to water-insoluble or poorly water-soluble molecules molecules of this invention renders them water-soluble and lowers their toxicity.

### BRIEF SUMMARY OF THE INVENTION

This invention relates to soluble derivatives of wortmannin that utilize water-soluble polymers as carriers for a drug.

In accordance with this invention there is provided a water-soluble drug-polymer conjugate wherein P is a water-soluble polymer; D is a wortmannin derivative; and X is a covalent linkage between the water-soluble polymer and a wortmannin derivative.

In one embodiment of the invention a wortmannin derivative utilizing water-soluble polymers has the structure of formula I

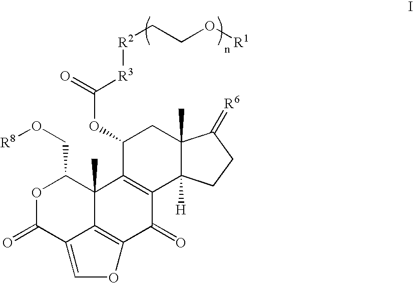

wherein:

R¹ is alkyl, or a drug-polymer conjugate of formula (A)

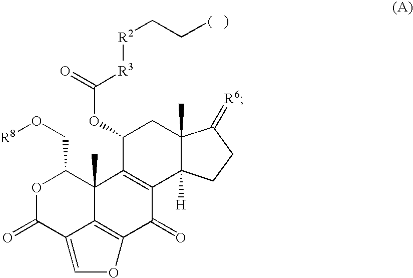

R² is —O—, —NH—, or —S—;

R³ is alkyl, a cycloalkyl, or aryl;

R⁶ is ═O or OR⁷;

R⁷ is H, COR⁹ or alkyl;

R⁸ is alkyl or H;

R⁹ is alkyl, H, aryl, or —CH₂Ar; and

n is 1-1000.

In one embodiment of the invention a wortmannin derivative utilizing water-soluble polymers has the structure of formula I

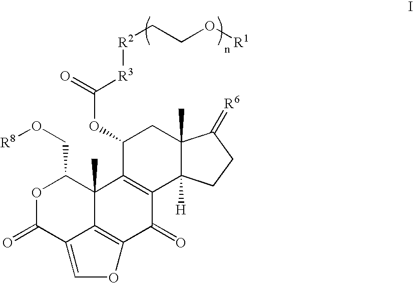

wherein:

R¹ is alkyl, or a drug-polymer conjugate of formula (B)

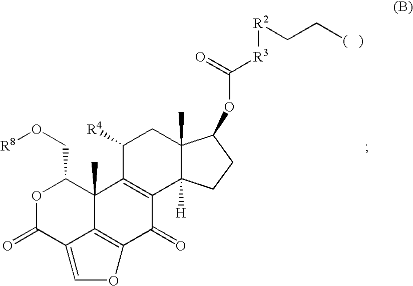

R² is —O—, —NH—, or —S—;

R³ is alkyl, a cycloalkyl, or aryl;

R⁴ is H, ═O, —O—COC₄H₉, or OR⁷;

R⁶ is ═O or OR⁷;

R⁷ is H, COR⁹ or alkyl;

R⁸ is alkyl or H;

R⁹ is alkyl, H, aryl, or —CH₂Ar; and

n is 1-1000.

In another embodiment of the invention a wortmannin derivative utilizing water-soluble polymers has the structure of formula II

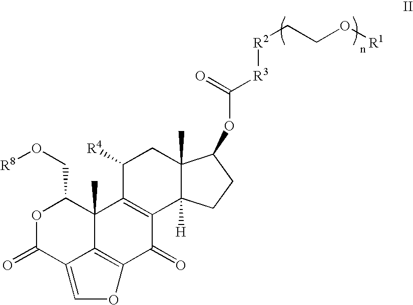

wherein:

R¹ is alkyl, or a water-soluble drug-polymer conjugate of formula (B)

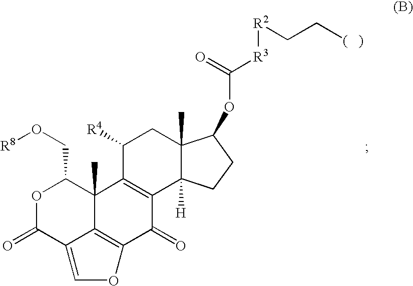

R² is —O—, —NH—, or —S—;

R³ is alkyl, a cycloalkyl, or aryl;

R⁴ is H, ═O, —O—COC₄H₉, or OR⁷;

R⁷ is H, COR⁹ or alkyl;

R⁸ is alkyl or H;

R⁹ is alkyl, H, aryl, or —CH₂Ar; and

n is 1-1000.

In one embodiment of the invention a wortmannin derivative utilizing water-soluble polymers has the structure of formula III:

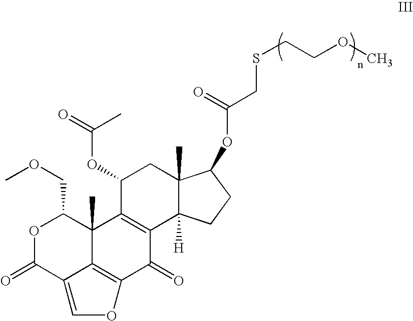

n is 1-1000.

In one embodiment of the invention a wortmannin derivative utilizing water-soluble polymers has the structure of formula IV:

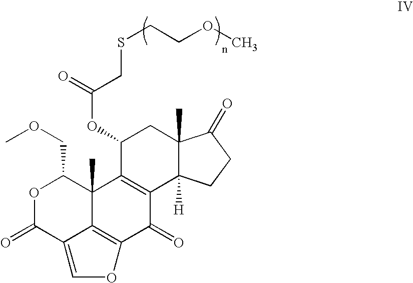

wherein n=1-1000.

When used herein the moiety

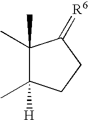

represents the structure:

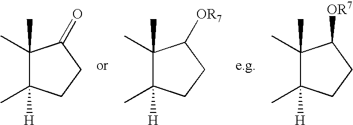

When used herein the moiety

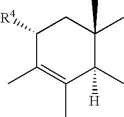

represents the structure:

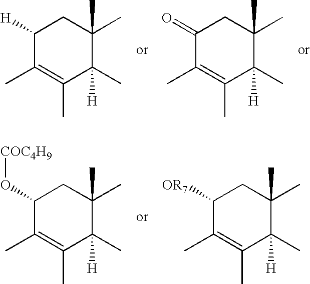

In an additional embodiment of this invention a process for the preparation of a water-soluble drug-polymer conjugate of formula (III) comprising:

a) adding a solvent to 17-dihydro-17-(1-iodoacetyl)-wortmannin to obtain a solution;

b) adding tertiary amine or sodium bicarbonate to the solution;

c) adding mPEG-sulfhydryl 5000 to the solution of step (b);

d) stirring the solution of step (c) for 30 minutes;

e) adding ether to the stirred solution;

f) collecting the solid; and

g) washing the collected solid with ether to obtain the pegylated wortmannin derivative,

is disclosed.

In an additional embodiment of this invention a process for the preparation of a water-soluble drug-polymer conjugate of formula (IV) comprising:

h) adding a solvent to 11-desacetyl-11-(1-iodoacetyl)-wortmannin to obtain a solution;

i) adding a tertiary amine or sodium bicarbonate to the solution;

j) adding mPEG-sulfhydryl 5000 to the solution of step (b);

k) stirring the solution of step (c) for 30 minutes;

l) adding ether to the stirred solution;

m) collecting the solid; and

n) washing the collected solid with ether to obtain the pegylated wortmannin derivative,

is disclosed.

In an embodiment of this invention a water-soluble drug-polymer conjugate has the structure of formula V:

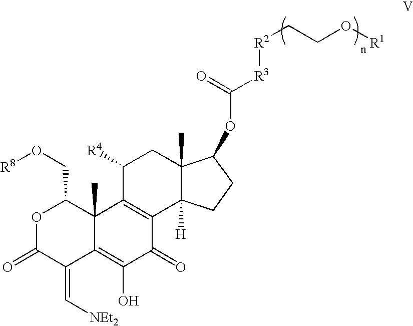

wherein:

R¹ is alkyl, or a drug-polymer conjugate of a single non-repeating formula (V)

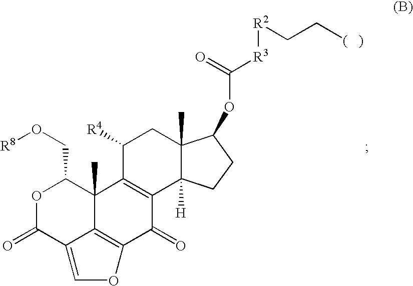

R² is —O—, —NH—, or —S—;

R³ is alkyl, a cycloalkyl, or aryl;

R⁴ is H, ═O, —O—COC₄H₉, or OR⁷;

R⁷ is H, COR⁹ or alkyl;

R⁸ is alkyl or H;

R⁹ is alkyl, H, aryl, or —CH₂Ar; and

n is 1-1000.

Another embodiment of this invention includes a process for the preparation of a compound of formula (V) comprising addition of amine to a compound of formula (I, II, III and IV) to obtain a compound of formula (V) or corresponding ring open structure. In a preferred embodiment the amine is diethyl amine.

The invention also provides a process for the preparation of a conjugate as described above which comprises reacting a compound of formula

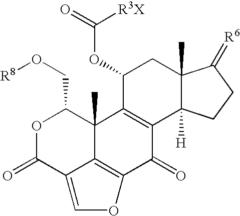

or a compound of formula

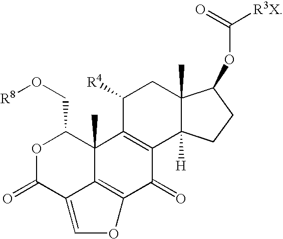

wherein:

R³ is alkyl, a cycloalkyl, or aryl;

R⁴ is H, ═O, —O—COC₄H₉ or OR⁷;

R⁶ is ═O or OR⁷;

R⁷ is H, COR⁹ or alkyl;

R⁸ is alkyl or H;

R⁹ is alkyl, H, aryl, or —CH₂Ar; and

n is 1-1000,

and X is a halogen, e.g. Br, Cl or I,

with a compound of formula

HR²—(CH₂CH₂O)ₙ—R¹

or a compound of formula

HR²—(CH₂CH₂O)ₙ—R²H

wherein R² is O, NH, or S;

n is 1-1000 and

R¹ is alkyl, or a drug-polymer conjugate of formula (A)

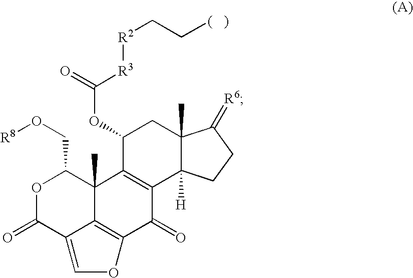

or a drug-polymer conjugate of formula (B)

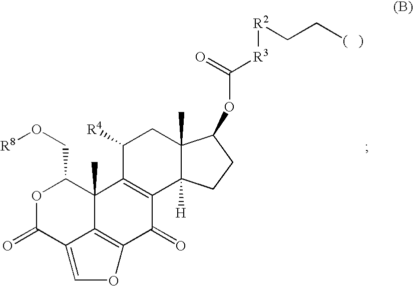

and R², R³, R⁴, R⁶, R⁸ and n are as defined above,

to provide the desired conjugate.

The invention further provides a process for the preparation of a compound or polymer of formula P—X-D:

wherein:

P is a water-soluble polymer;

D is a wortmannin derivative; and

X is a covalent linkage between a water-soluble polymer and the wortmannin derivative,

which comprises reacting a polymer of formula P—XH, wherein P and X are as defined above,

with a compound of formula DX, wherein X is a halogen, e.g. Br, Cl or I,

to provide the desired product.

The conjugates of the invention are preferably water soluble conjugates, more preferably water soluble drug-polymer conjugates.

### BRIEF DESCRIPTION OF THE SEVERAL VIEWS OF THE DRAWING

FIG. 1 represents antitumor activity of pegylated-17-hydroxy-wortmannin versus unpegylated-17-hydroxy-wortmannin. A tumor cell line for brain tumor, PTEN (−/−) U87MG Glioma was implanted in mice. The mice were dosed IV on days 0-4. The graph represents relative tumor growth (y-axis) (1, 1.5, 2, 2.5, 3, 3.5, and 4 mm) at doses of vehicle, 15 mg/kg, 5 mg/kg, 1.5 mg/kg, 0.5 mg/kg pegylated-17-hydroxy-wortmannin, 1 mg/kg, and 0.5 mg/kg unpegylated-17-hydroxy-wortmannin (x-axis).

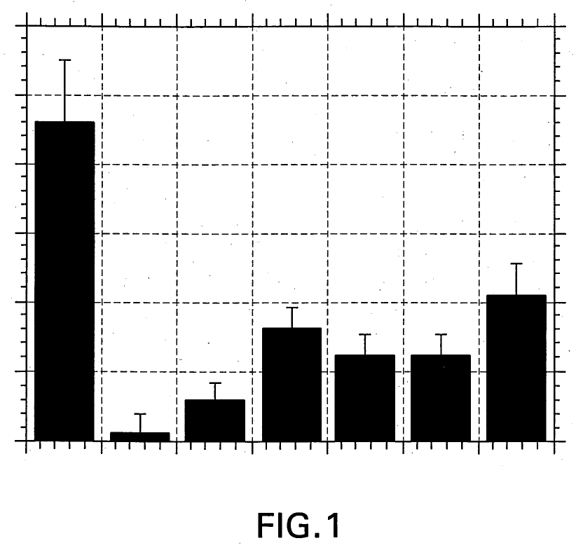

FIG. 2 represents in vivo antitumor activity of a wortmannin derivative against PTEN (−/−) U87MG glioma. In this experiment, U87MG glioma growing as subcutaneous xenografts in nude mice were staged on day 0, and dosed on days 0-4 at 0.15, 0.5, 1.5, 5 and 15 mg/kg/dose of a wortmannin derivative of this invention. As shown in FIG. 2, a minimally efficacious dose (MED) was 0.5 mg/kg/dose (MED), which achieved a 50% inhibition of tumor growth on day 7. A dose dependent further increase of anticancer activity was evident. The maximal tolerated dose (MTD) in this experiment was 15 mg/kg/dose.

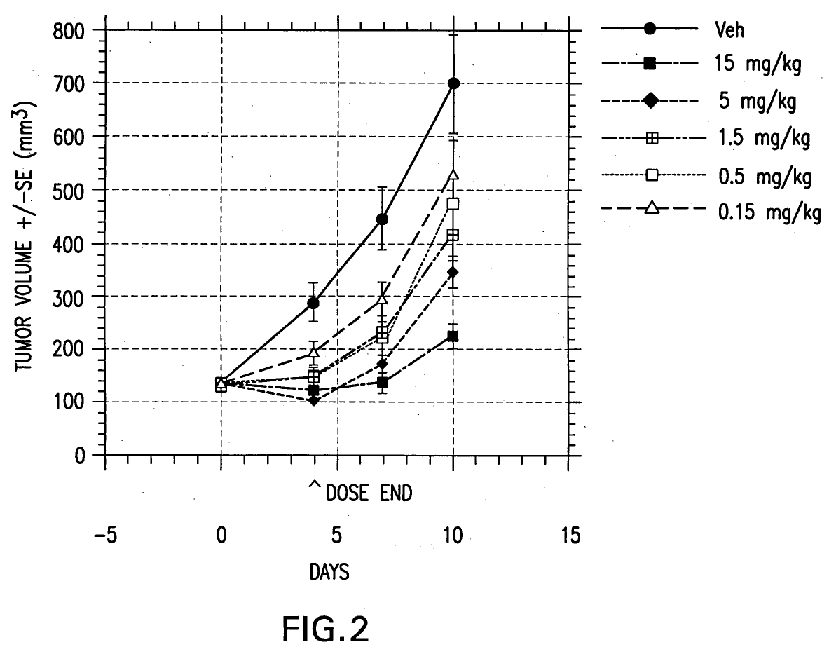

FIG. 3 represents a combination of antitumor activity of a wortmannin derivative and paclitaxel in U87MG glioma model. In the U87MG glioma study shown in FIG. 3, a wortmannin derivative of this invention was dosed IV on days 0-4. Paclitaxel was dosed IP on days 0 and 7. The MTD of paclitaxel is 60 mg/kg/dose following a weekly dosing schedule. Mice were treated with a wortmannin derivative of this invention at 1 mg/kg/dose, paclitaxel at 30 and 60 mg/kg/dose, or treated in combination with a wortmannin derivative of this invention at 1 mg/kg/dose and paclitaxel at 30 mg/kg/dose. A wortmannin derivative of this invention alone was equally active as the 30 mg/kg/dose of paclitaxel. Combination of both agents was more efficacious than either agent alone. The tumor suppression in the combination group was similar to that achieved by 60 mg/kg/dose of paclitaxel.

FIG. 4 represents pooled data from two experiments using NSCLC A549 model. A wortmannin derivative was dosed IV on days 0-4, 14-18. Paclitaxel was dosed IP on days 0, 7 and 14. Mice were treated with a wortmannin derivative at 5 mg/kg/dose, paclitaxel at 30 mg/kg/dose, or treated in combination of the two. A wortmannin derivative alone at 5 mg/kg/dose was similarly active as 30 mg/kg/dose of paclitaxel. It is evident that the combination treatment produced a most interesting antitumor activity, in which a complete arrest of tumor growth was achieved.

FIG. 5 represents an assessment of the combination antitumor activity with pegylated-rapamycin (Peg-rapa), a potent inhibitor of TOR, in U87MG glioma model. A wortmannin derivative at 1 mg/kg/dose and Peg-rapa at 0.1 mg/kg/dose were dosed IV either alone or in combination on days 0-4. The data in FIG. 5 indicated that the combination treatment clearly produced a better antitumor activity than either agent alone.

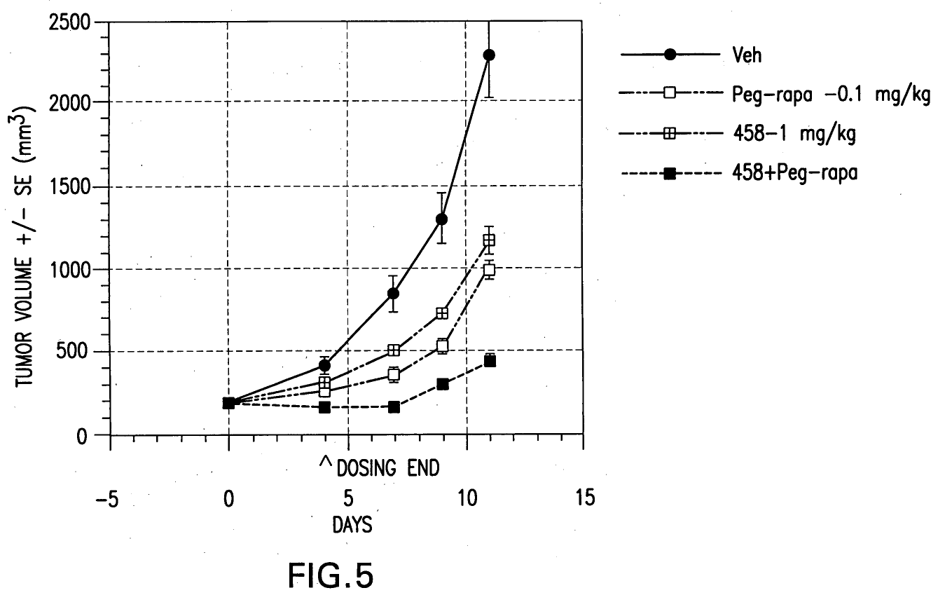

### DETAILED DESCRIPTION OF THE INVENTION

The following experimental details are set forth to aid in an understanding of the invention, and are not intended, and should not be construed, to limit in any way the invention set forth in the claims that follow thereafter.

The current invention concerns the discovery of wortmannin derivatives utilizing water-soluble polymers.

The present invention relates to water-soluble drug polymers. Water-soluble polymers having the structure Polyethylene glycol (PEG) are linear or branched, neutral polymers available in a variety of molecular weights and are soluble in water and most organic solvents. At molecular weights less than 1000, PEGs are a viscous, colorless liquid, at higher molecular weight PEGs are waxy, white colloids. The melting point of the solid is proportional to the molecular weight, approaching a plateau at 67° C. Molecular weights range from a few hundred to approximately 80,000. Examples of water-soluble polymers that may be used in enhancing deliverability of drugs include for example polyethylene glycols (PEG), PEG methyl ether, PEG-block-PEG-block-PEG, polyvinyl alcohol, polyhydroxyethyl, polymethacrylate, polyacrylamide, polyacrylic acid, polyethyloxazoline, polyvinyl pyrrolidinone, and polysaccharides. In a preferred embodiment of this invention water-soluble polymers containing 1 to 1000 monomers are attached to the wortmannin derivatives, with a more preferred number of monomers being 250 to 400, and the most preferable number being 50 to 150. The molecular weight of the attached water-soluble polymers can range from about 400 to about 80,000. In a preferred embodiment the molecular weight is in the range of about 1000 to about 8000 with the most preferred embodiment being in a range of about 4000 to about 6000.

The water-soluble polymer is attached to the wortmannin derivative by a covalent linkage. The covalent linkage can be by means of an ester, diester, urethane, amide, secondary or tertiary amine, ether, or any covalent linkage that enables the delivery of a water-insoluble or poorly water-soluble drug in a soluble form into the body of a mammal.

The wortmannin derivatives of this invention include water-soluble drug-polymer conjugates having the following structures:

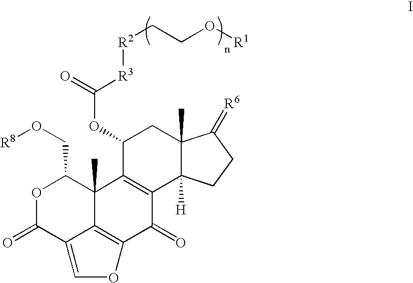

wherein:

R¹ is alkyl, or a drug-polymer conjugate of formula (A)

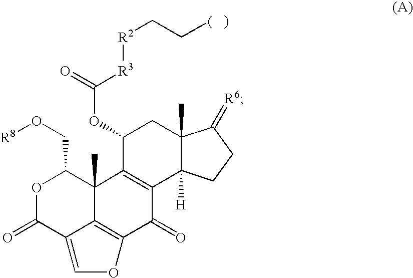

R² is —O—, —NH—, or —S—;

R³ is alkyl, a cycloalkyl, or aryl;

R⁶ is ═O or OR⁷;

R⁷ is H, COR⁹ or alkyl;

R⁸ is alkyl or H;

R⁹ is alkyl, H, aryl, or —CH₂Ar; and

n is 1-1000.

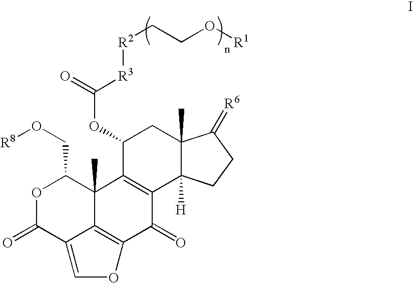

wherein:

R¹ is alkyl, or a drug-polymer conjugate of formula (B)

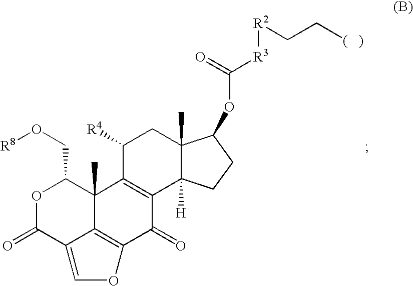

R² is —O—, —NH—, or —S—;

R³ is alkyl, a cycloalkyl, or aryl;

R⁴ is H, ═O, —O—COC₄H₉, or OR⁷;

R⁶ is ═O or OR⁷;

R⁷ is H, COR⁹ or alkyl;

R⁸ is alkyl or H;

R⁹ is alkyl, H, aryl, or —CH₂Ar; and

n is 1-1000.

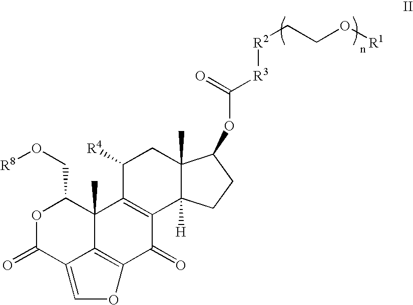

wherein:

R¹ is alkyl, or a drug-polymer conjugate of formula (B)

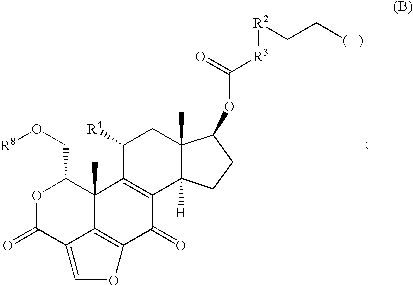

R² is —O—, —NH—, or —S—;

R³ is alkyl, a cycloalkyl, or aryl;

R⁴ is H, ═O, —O—COC₄H₉, or OR⁷;

R⁷ is H, COR⁹ or alkyl;

R⁸ is alkyl or H;

R⁹ is alkyl, H, aryl, or —CH₂Ar; and

n is 1-1000.

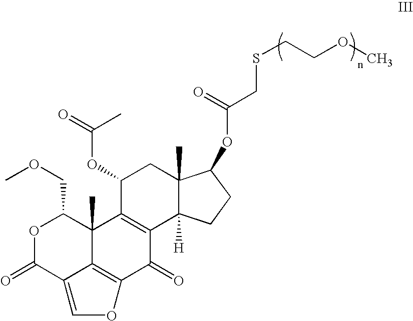

n is 1-1000.

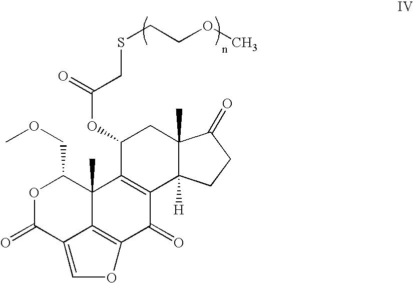

wherein n=1-1000.

For purposes of this invention the term “alkyl” includes both straight and branched alkyl moieties and may be substituted or unsubstituted, preferably of 1 to 8 carbon atoms. The term “cycloalkyl” refers to alicyclic hydrocarbon groups having 3 to 12 carbon atoms and includes but is not limited to: cyclopropyl, cyclobutyl, cyclopentyl, cyclohexyl, cycloheptyl, or norbornyl.

For purposes of this invention the term “aryl” or “Ar” is defined as an aromatic hydrocarbon moiety and may be substituted or unsubstituted. An aryl may be selected from but not limited to a phenyl group.

For purposes of this invention “acyl” is a radical of the formula —(C═O)-alkyl or —(C═O)-perfluoroalkyl wherein the alkyl radical or perfluoroalkyl radical is 1 to 7 carbon atoms; preferred examples include but are not limited to, acetyl, propionyl, butyryl, trifluoroacetyl.

For purposes of this invention a “solvent” is a polar compound in which PEGSH can dissolve and includes for example dioxane, acetonitrile, tetrahydrofuran (THF), or Dimethylformide (DMF).

For purposes of this invention a “tertiary amine” includes for example N,N-diisopropylethylamine, triethylamine, tributylamine.

For purposes of this invention amine that is not a tertiary amine, can include but is not limited to alkyl, heteroaryl, aryl, piperidine, piperazine, di-amino propane, amino acids, or any primary or secondary amine.

R¹ is preferably methyl. R² is preferably S. R³ is preferably —CH₂— or —H₂—CH₂—. R⁴ is preferably —OR⁷. R⁶ is preferably =0. R⁷ is preferably CO⁹. R R⁸ is preferably methyl. R⁹ is preferably methyl. One embodiment of this invention comprises compounds wherein R¹ is methyl; R² is S; R³ is —CH₂— or —CH₂—CH₂—; R⁴ is —OR⁷; R⁷ is —COR⁹; R⁸ is methyl and R⁹ is methyl. A further embodiment of the invention comprises compounds wherein R¹ is methyl; R² is S; R³ is —CH₂— or —CH₂—CH₂—; R⁶ is 0 and R is methyl. n is 1-1000, preferably 50-400, including the ranges 50-150 and 250-400.

In one embodiment of this invention the substituted aryl may be optionally mono, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, —SO₃H, —SO₂NH₂, —SO₂NHalkyl, —SO₂N(alkyl)₂, —CO₂H, CO₂NH₂, CO₂NHalkyl, and —CO₂N(alkyl)₂.

In one embodiment of this invention the substituted aryl may be optionally mono, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, —SO₃H, —SO₂NH₂, —SO₂NHalkyl, —SO₂N(alkyl)₂, —CO₂H, CO₂NH₂, CO₂NHalkyl, and —CO₂N(alkyl)₂.

In another embodiment, the present invention provides a method for the treatment or prevention of a pathological condition or disorder mediated in a mammal. The present invention accordingly provides to a mammal, a pharmaceutical composition that comprises a water-soluble drug-polymer conjugate of this invention in combination or association with a pharmaceutically acceptable carrier. The water-soluble drug-polymer conjugate of this invention may be administered alone or in combination with other therapeutically effective compounds or therapies for the treatment or prevention of a pathological condition or disorder mediated in a mammal.

When treating or inhibiting a pathological condition or disorder mediated in a mammal the water-soluble drug-polymer conjugates are preferably provided orally or subcutaneously. The water-soluble drug-polymer conjugates may be provided by intralesional, intraperitoneal, intramuscular or intravenous injection; infusion; liposome-mediated delivery; topical, nasal, anal, vaginal, sublingual, uretheral, transdermal, intrathecal, ocular or otic delivery. In order to obtain consistency in providing the water-soluble drug-polymer conjugate of this invention it is preferred that a water-soluble drug-polymer conjugate of the invention is in the form of a unit dose. Suitable unit dose forms include tablets, capsules and powders in sachets or vials. Such unit dose form may contain from 0.1 to 100 mg of a wortmannin derivative conjugated to a water-soluble drug-polymer of the invention and preferably from 2 to 50 mg. Still further preferred unit dosage forms contain 5 to 25 mg of a wortmannin derivative coupled to a water-soluble drug-polymer of the present invention. The water-soluble drug-polymer conjugates of the present invention can be administered orally at a dose range of about 10 to 1000 mg/kg or preferably at a dose range of 0.5 to 10 mg/kg. Such water-soluble drug-polymer conjugates may be administered from 1 to 6 times a day, more usually from 1 to 4 times a day. The effective amount will be known to one of skill in the art; it will also be dependent upon the form of the water-soluble drug-polymer conjugate. One of skill in the art could routinely perform empirical activity tests to determine the bioactivity of the water-soluble drug-polymer conjugate in bioassays and thus determine what dosage to administer.

The water-soluble drug-polymer conjugates of the invention may be formulated with conventional excipients, such as a filler, a disintegrating agent, a binder, a lubricant, a flavoring agent, a color additive, or a carrier. The carrier may be for example a diluent, an aerosol, a topical carrier, an aqueous solution, a nonaqueous solution or a solid carrier. The carrier may be a polymer or a toothpaste. A carrier in this invention encompasses any of the standard pharmaceutically accepted carriers, such as phosphate buffered saline solution, acetate buffered saline solution, water, emulsions such as an oil/water emulsion or a triglyceride emulsion, various types of wetting agents, tablets, coated tablets and capsules.

When provided orally or topically, such water-soluble drug-polymer conjugates would be provided to a subject by delivery in different carriers. Typically, such carriers contain excipients such as starch, milk, sugar, certain types of clay, gelatin, stearic acid, talc, vegetable fats or oils, gums, or glycols. The specific carrier would need to be selected based upon the desired method of delivery, for example, phosphate buffered saline (PBS) could be used for intravenous or systemic delivery and vegetable fats, creams, salves, ointments or gels may be used for topical delivery.

The water-soluble drug-polymer conjugates of the present invention may be delivered together with suitable diluents, preservatives, solubilizers, emulsifiers, adjuvants and/or carriers useful in treatment or prevention of pathological condition or disorder mediated in a mammal. Such compositions are liquids or lyophilized or otherwise dried formulations and include diluents of various buffer content (for example, Tris-HCl, acetate, phosphate), pH and ionic strength, additives such as albumins or gelatin to prevent absorption to surfaces, detergents (for example, TWEEN 20, TWEEN 80, PLURONIC F68, bile acid salts), solubilizing agents (for example, glycerol, polyethylene glycerol), anti-oxidants (for example, BHA and BHT, ascorbic acid, sodium metabisulfate), preservatives (for example, thimerosal, benzyl alcohol, parabens), bulking substances or tonicity modifiers (for example, lactose, mannitol), covalent attachment of polymers such as polyethylene glycol, complexation with metal ions, or incorporation of the water-soluble drug-polymer conjugate into or onto particulate preparations of hydrogels or liposomes, micro-emulsions, micelles, unilamellar or multilamellar vesicles, erythrocyte ghosts, or spheroplasts. Such compositions will influence the physical state, solubility, stability, rate of in vivo release, and rate of in vivo clearance of the water-soluble drug-polymer conjugate or composition. The choice of compositions will depend on the physical and chemical properties of the water-soluble drug-polymer conjugate capable of treating or inhibiting a pathological condition or disorder mediated in a mammal.

The water-soluble drug-polymer conjugate of the present invention may be delivered locally via a capsule that allows a sustained release of the water-soluble drug-polymer conjugate over a period of time. Controlled or sustained release compositions include formulation in lipophilic depots (for example, fatty acids, waxes, oils).

For purposes of this invention a pathological condition or disorder mediated in a mammal includes any condition that expresses PI 3 kinase and/or TOR kinase at levels greater than that found in a healthy mammal. The water-soluble drug-polymer conjugates of this invention are used as inhibitors of PI 3 kinase and TOR kinase. The pathological condition or disorder mediated in a mammal for which inhibitors of PI 3 kinase and TOR kinase have been effective in treating or inhibiting are iscaemic heart disease, restenosis, inflammation, platelet aggregation, sclerosis, respiratory disorders, HIV, bone resorption, non-small cell lung cancer, and brain cancer.

The compounds of this invention may be provided as a single compound or in combination with other compounds.

Inhibition of PI3K might be expected to enhance therapeutic activity of other agents that modulate growth factor signaling, cytokine response and cell cycle control. Wortmannin derivatives synergize with interferon-α in causing tumor regression and enhancing anticancer activity of pegylated-rapamycin, a specific inhibitor of mTOR kinase.

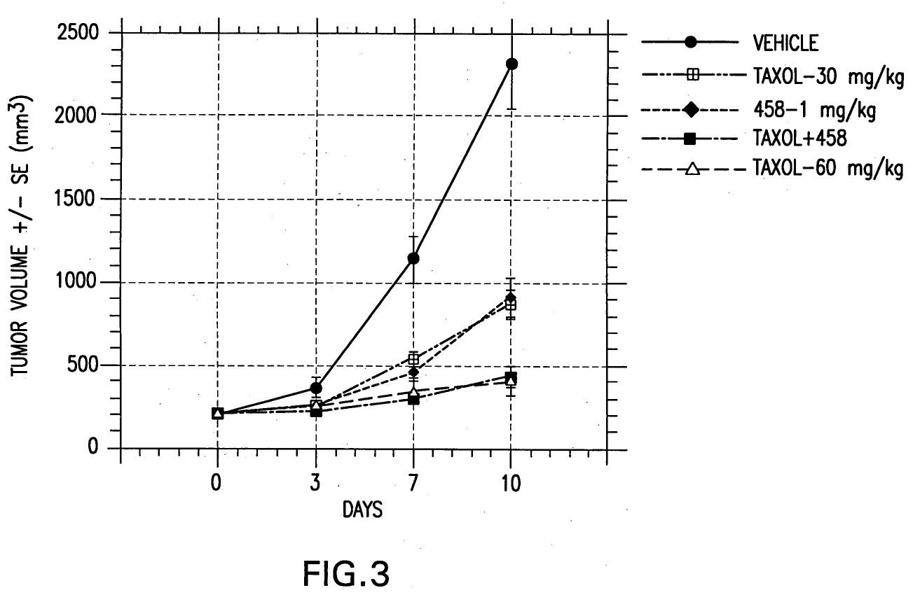

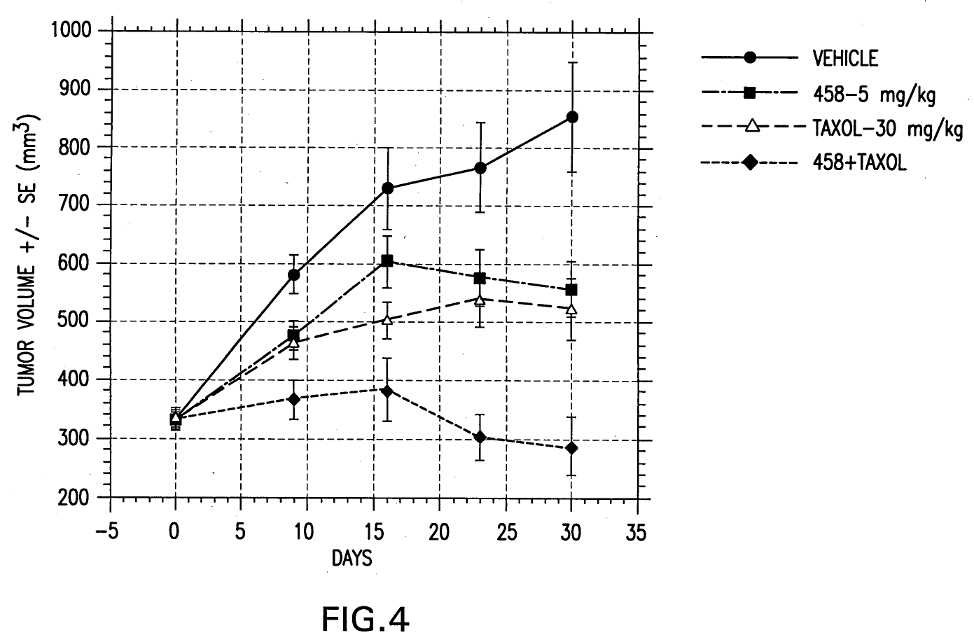

A cellular inhibition of PI3K or AKT leads to a reduction in survival, a critical process underlying the anticancer activity of many standard cancer therapies. However, in many cases, tumor cells rapidly develop chemo-resistance. One cellular mechanism of resistance relates to constitutive elevation of PI3K/AKT pathway. Thus, combination treatment of cytotoxics with an inhibitor of PI3K may further augment efficacy in an initial therapy and may also help in a restoration of chemo-sensitivity in recurring therapies. Wortmannin derivatives are shown to potentiate paclitaxel anticancer efficacy in lung cancer and in glioma. (See FIGS. 3 and 4.)

Preparation of 17-dihydro-17-(1-iodoacetyl)-wortmannin

1,3-Dicyclohexylcarbodiimide (DCC), 4-Dimethylaminopyridine and wortmannin were purchased from Aldrich Chemical Co. (Milwaukee, Wis.). Methoxy-PEG-SH of average molecular weight 5000 (mPEG-SH 5000) was purchased from Shearwater Polymers, Inc. (Huntsville, Ala.). All solvents were HPLC grade and all other chemicals were analytical reagent grade or equivalent. The preparative High Performance Liquid Chromatography (HPLC) consisted of two Dynamax solvent delivery systems (Model SD-1) and one Dynamax absorbance detector (Model UV-1) from Rainin Instrument Inc. (Woburn, Mass.). Additional equipment included an automatic speed-vac concentrator (Savant, Model AS 160) from Savant Instruments, Inc. (Holbrook, N.Y.) and a BUCHI rotary evaporation system (RE 260 and R 124) from Buchi (Flawil, Switzerland). ¹H-NMR spectra were recorded on a 400 MHz NMR spectrophotometer using CDCl₃ as solvents.

HPLC method-Preparative HPLC was run on a Prep Nova-pak HR C18 column (300×19 mm from Waters) using gradient method that held 80% A and 20% B for the first 5 minutes, 80% A and 20% B to 30% A and 70% B in 30 minutes. Buffer A was 90% water and 10% acetonitrile. Buffer B was 10% water and 90% acetonitrile. The flow rate was 20 mL/minute, UV at 254 nm. The fraction at 27 minutes (water-soluble drug-polymer conjugate III) or at 15 minutes (water-soluble drug-polymer conjugate IV) was collected and extracted with methylene chloride and worked-up. The fraction collected from HPLC was extracted with methylene chloride. The organic layer was dried with anhydrous sodium sulfate and worked up as follow. The fraction collected from HPLC was extracted with methylene chloride. The organic layer was dried with anhydrous sodium sulfate. The organic solvent was removed using a rotary evaporation system. The residual was transferred into small vial and was dried in the speed-vac overnight.

A solution of 60 mg wortmannin (0.14 mmol from Aldrich) in 12 mL tetra-hydrofuran (THF) was cooled in a 0° C. ice bath under nitrogen. 1M borane in THF solution (134 μL, 0.14 mmol from Aldrich) was added and the reaction mixture was stirred at 0° C. for 3.5 hours. The reaction was quenched with 1 mL water. After warming to room temperature, the reaction mixture was diluted with water and extracted with ethyl acetate. After work up, about 60 mg (90% pure 17-hydroxy-wortmannin by HPLC) solid was obtained. This solid (about 0.126 mmol 17-hydroxy-wortmannin) was dissolved in 15 mL methylene chloride, reacted with iodoacetic acid (24 mg, 0.13 mmol), dicyclohexylcarbodiimide (DCC) (27 mg, 0.13 mmol) and 4-N,N-dimethylaminopyridine (DMAP) (0.1 mg as catalyst). The reaction mixture was kept at room temperature for 1 hour. After work up, about 75 mg crude product (yellow solid) was obtained. Pure 17-dihydro-17-(1-iodoacetyl)-wortmannin was isolated by preparative HPLC. A total of 54 mg of white solid was obtained. [M+H] at m/z 599 and [M+NH₄] at m/z 616, exact mass of [M+H] ion: 599.0758 Da, calculated mass of C₂₅H₂₈O₉I: 599.0772 Da. ¹H-NMR (CDCl₃) δ 0.94 (s, 3H), 1.54 (dd, J=12.16, 10.06, 1H), 1.69 (m, 1H), 1.69 (m, 3H), 1.78 (m, 1H), 2.15 (s, 3H), 2.31 (m, 1H), 2.56 (dd, J=12.16, 7.36, 1H), 2.63 (ddd, J=2.7, 1H), 2.85 (ddd, J=20.12, 9.91, 2.7, 1H), 2.99 (dd, J=11.11, 7.21, 1H), 3.19 (s, 3H), 3.46 (dd, J=11.11, 1.8, 1H), 3.69 (d, J=10.6, 1H), 3.72 (d, J=10.6, 1H), 4.76 (dd, J=7.21, 1.8, 1H), 4.87 (dd, J=7.51, 9.46, 1H), 6.10 (ddd, J=10.06, 7.36, 3.0, 1H), 8.23 (s, 1H). ¹³C-NMR δ −5.62, 12.79, 21.14, 24.65, 26.58, 27.04, 40.11, 40.72, 44.07, 44.99, 59.44, 72.90, 88.88, 114.25, 141.11, 142.72, 144.93, 148.68, 149.84, 157.66, 168.94, 169.54, 172.77.

Preparation of Conjugate (III) of M-PEG-SH 5000 and 17-dihydro-17-(1-iodoacetyl)-wortmannin

40 mg (0.067 mmol) 17-dihydro-17-(1-iodoacetyl)-wortmannin was dissolved in 15 mL acetonitrile and 10 mL 0.1 M sodium bicarbonate under nitrogen. A total of 345 mg M-PEG-SH-5000 (0.069 mmol) was added within 1 hour (4 batches). After stirring another hour at room temperature, the reaction mixture was extracted with methylene chloride and worked-up. About 320 mg crude product was obtained. A total of 209 mg pure water-soluble drug-polymer conjugate III was obtained from 260 mg of crude product after prep-HPLC. ¹H-NMR (CDCl₃) δ 0.92 (s, 3H), 1.53 (dd, 1H), 1.68 (m, 1H), 1.75 (s, 3H), 1.77 (m, 1H), 2.14 (s, 3H), 2.32 (m, 1H), 2.53 (dd, 1H), 2.63 (s, 1H), 2.85 (overlap, 1H), 2.85 (t, J=6.56, 2H), 2.99 (dd, J=11.03, 7.3, 1H), 3.2 (s, 3H), 3.31 (s, 2H), 3.38 (s, 3H), 3.47 (dd, J=11.03, 1.79, 1H), 3.55 (s, 2H), 3.64 (m), 3.7 (s, 2H), 4.76 (dd, J=7.3, 1.79, 1H), 4.86 (dd, 1H), 6.15 (s, 1H), 8.24 (s, 1H). ¹³C-NMR δ 12.85, 21.11, 24.68, 26.48, 27.39, 34.01, 40.19, 40.68, 44.05, 44.81, 59.03, 59.42, 70.3, 70.35, 70.57, 70.88, 71.93, 72.88, 80.69, 88.86, 114.21, 141.19, 142.68, 144.92, 148.63, 149.88, 157.63, 169.58, 170.52, 172.75.

Preparation of 11-desacetyl-11-(1-iodoacetyl)-wortmannin

11-O-desacetylwortmannin (prepared from wortmannin, J. Med Chem, 1996, 39, 5021), 42 mg (0.11 mmol), was dissolved in 8 mL methylene chloride, reacted with iodoacetic acid (24 mg, 0.13 mmol), DCC (27 mg, 0.13 mmol) and DMAP (0.1 mg as catalyst). The reaction mixture was kept at room temperature for 2 hours. After work up, about 80 mg crude product (yellow solid) was obtained. Pure 11-desacetyl-11-(1-iodoacetyl)-wortmannin was isolated by preparative HPLC. A total of 41 mg of yellowish solid was obtained. [M+H] at m/z 555 and [M+NH₄] at 572, exact mass of [M+NH₄] ion: 572.0783 Da, calculated mass of C₂₃H₂₇O₈NI: 572.0775 Da. ¹H-NMR (CDCl₃) δ 0.97 (s, 3H), 1.66 (dd, J=12.84, 8.80 Hz, 1H), 1.75 (s, 3H), 2.06 (ddd, J=22.25, 12.72, 8.93 Hz, 1H), 2.27 (dt, J=19.68, 8.93 Hz, 1H), 2.65 (dd, J=12.84, 7.58 Hz, 1H), 2.61-3.18 (m, 2H), 2.92 (ddd, J=12.72, 5.99, 2.57 Hz, 1H), 3.01 proR (ddd, J=11.25, 6.72 Hz, 1H), 3.23 (s, 3H), 3.46 proS (dd, J=11.25, 1.59 Hz, 1H), 3.65 (d, J=9.9 Hz, 1H), 3.89 (d, J=9.9 Hz, 1H), 4.83 (dd, J=6.72, 1.59 Hz, 1H), 6.15 (ddd, J=8.8, 7.58, 2.57 Hz, 1H), 8.26 (s, 1H). ¹³C-NMR δ −6.68, 14.65, 22.93, 26.48, 35.05, 35.74, 40.87, 44.11, 49.04, 59.69, 71.84, 73.31, 88.54, 114.28, 140.92, 142.74, 144.81, 148.77, 150.09, 157.52, 167.8, 172.48, 215.89.

Preparation Conjugate (IV) of M-PEG-SH 5000 and 11-desacetyl-11-(1-iodoacetyl)-wortmannin

30 mg (0.054 mmol) 11-desacetyl-11-(1-iodoacetyl)-wortmannin was dissolved in 15 mL acetonitrile and 10 mL 0.1 M sodium bicarbonate under nitrogen. A total of 300 mg M-PEG-SH-5000 (0.060 mmol) was added within 1 hour (3 batch). After stirring another hour at room temperature, the reaction mixture was extracted with methylene chloride and worked-up. About 274 mg crude product was obtained. A total of 172 mg pure water-soluble drug-polymer conjugate IV was obtained after prep-HPLC. ¹H-NMR (CDCl₃) δ 0.98 (s, 3H), 1.64 (dd, J=12.88, 8.87, 1H), 1.74 (s, 3H), 2.06 (ddd, J=22.25, 12.72, 9.03, 1H), 2.27 (dd, J=19.58, 9.37, 1H), 2.6 (dd, J=19.58, 8.53, 1H), 2.63 (dd, J=12.88, 7.53, 1H), 2.84 (t, J=6.36, 2H), 2.91 (ddd, J=12.72, 5.86, 2.68, 1H), 3.01 proR (dd, J=11.38, 6.36, 1H), 3.16 (s, 3H), 3.19 (m, 1H), 3.38 (s, 3H), 3.46 proS (dd, J=11.38, 6.36, 1H), 3.55 (s, 2H), 3.65 (m), 3.7 (s, 2H), 3.34 (d, J=9.87, 2H), 4.91 (dd, J=6.36, 1.84, 1H), 6.15 (ddd, J=8.87, 7.53, 2.68, 1H), 8.27 (s, 1H). ¹³C-NMR δ 14.6, 22.93, 26.51, 31.99, 33.64, 35.72, 35.76, 40.82, 44.08, 49.1, 59.02, 59.47, 70.36, 70.55, 70.87, 71.18, 71.92, 73.05, 88.35, 114.35, 140.52, 142.97, 144.74, 149.08, 150.07, 157.68, 169.02, 172.52, 215.97.

### Synthesis of PEG 11-Hydroxywortmannin

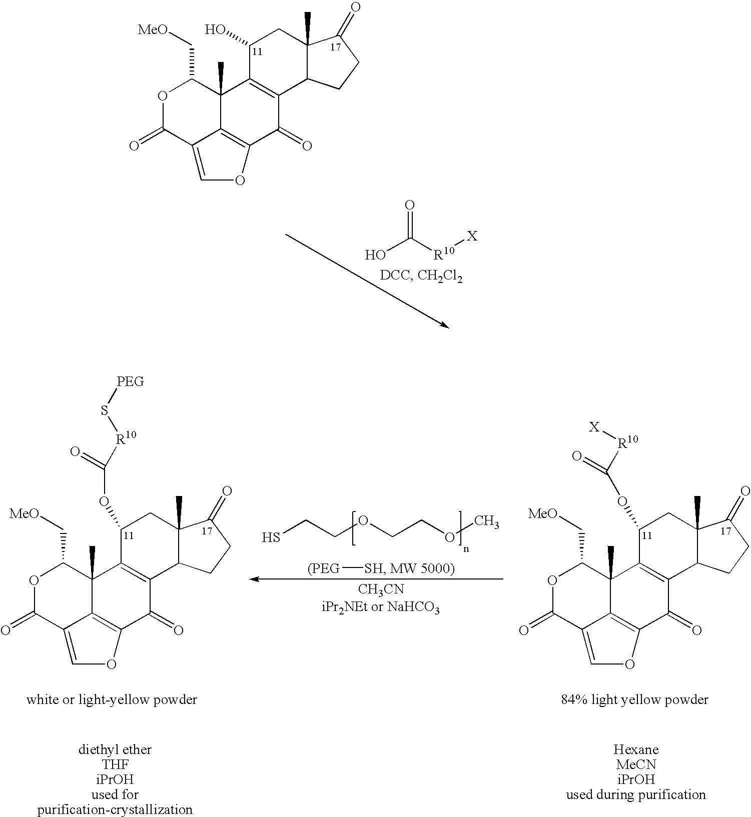

X is Br, Cl, or I and R¹⁰ is (CH2)ₙ or

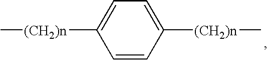

where n=0-5.

### Synthesis of PEG 17-Hydroxywortmannin

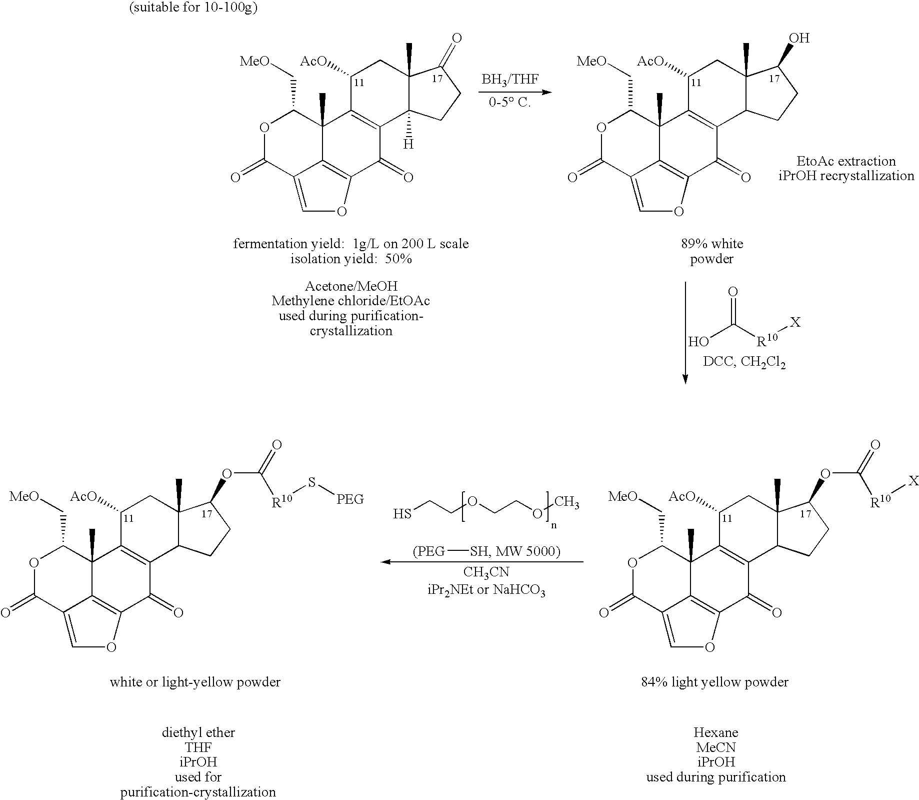

X is Br, Cl, or I and R¹⁰ is (CH₂)ₙ or

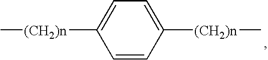

where n=0-5.

Alternate Method for preparation of conjugate (III) of mPEGSH 5000 and 17-dihydro-17-(1-iodoacetyl)-wortmannin (pegylated wortmannin derivative)

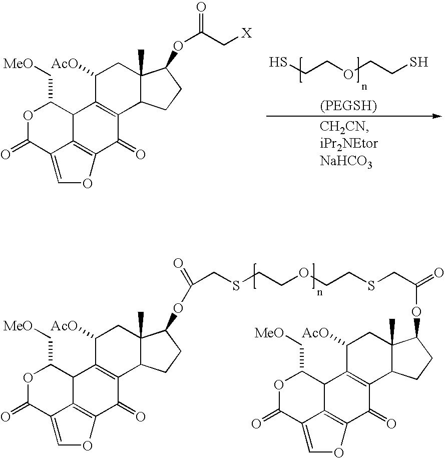

Preparation of Conjugate V

To a solution of conjugate III (n=100-110) (3 g) in dichloromethane (12 mL) was added diethylamine (200 uL). After 18 h the volatiles were removed in vacuo. The resulting yellow solid was dissolved in a minimum of dicloromethane. Diethyl ether was added and the resulting yellow powder was collected by filtration. The title compound was obtained as a yellow powder (2.8 g). Mass spectra m/z: calculated for n=109; 5526, found=5526.

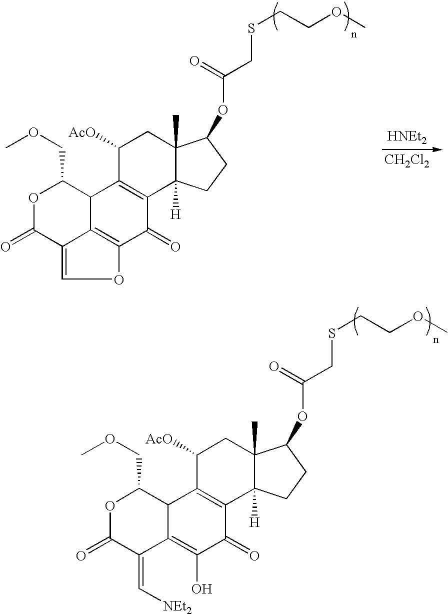

To a solution of 17-dihydro-17-(1-iodoacetyl)-wortmannin (215 mg, 0.36 mmol) in acetonitrile (20 mL) was added N,N-diisopropylethylamine (150 mg, 1.16 mmol), followed by PEG-(sulfhydryl)₂ 5000 (PEGSH, 780 mg). The mixture was then stirred for 30 minutes and ether (400 mL) was added, the solid was collected on the Buchner funnel and washed with ether, the PEG-di-wortmannin conjugate product was obtained as an off white solid.

In Vivo Xenograft Studies

Balb/c nu/nu (athymic) mice were housed in accordance with Association for Accreditation of Laboratory Animal Care (AALAACC) standards for at least one week prior to their experimental usage. The animals were housed in microisolator cages and handled only in a laminar flow hood. All food and water was autoclaved. Mice were inoculated on the left flank with a volume of 200 μL using a 25-26 gauge sterile needle and syringe with a suspension of cells. The cells were resuspended in full growth media and delivered at 10 million cells per mouse. When the resulting tumors reached the appropriate size for staging, the mice were regrouped to produce equivalent sized groups with n=10. Once staged, the mice were dosed 0.2 cc iv with the water-soluble drug-polymer conjugate II and water-soluble drug-polymer conjugate IV resuspended in sterile, distilled water. Wortmannin and other non-pegylated water-soluble drug-polymer conjugate were prepared 10 mg/ml in Dimethyl sulfoxide (DMSO) and diluted with Phosphate Buffered Saline (PBS) right before injecting into the mouse. Treatment was administered as a daily×5 dosing schedule repeated every 2 weeks until the tumors reach 10% of the animal's weight. The growth of the solid tumor was monitored twice a week for the duration of the experiment. Tumor size was quantitated using sliding vernier calipers and the mass was calculated using the formula L×W divided by 2 in mm. Conversion from cubic mm to mg was made assuming unit density. Tumors were not allowed to grow larger than 15% of the mouse's weight, at which point the mouse was euthanized.

Anticancer activity in xenograft U87MG glioblastoma model

| | Dose | IV Dosing | | Tumor Volume (mm³) | | | | | |
|---|---|---|---|---|---|---|---|---|---|
| Group | (mg/kg) | Schedule | | day 0 | day 4 | day 7 | day 11 | day 14 | day 17 |
| Vehicle | | d 0-4 | mean | 135.7 | 283.9 | 322.6 | 585.1 | 1100.2 | 2122.4 |
| | | | se | 12.8 | 28.2 | 44.9 | 106.4 | 215.3 | 321.8 |
| Water-soluble | 0.7 mg/kg | d 0-4 | mean | 131.0 | 165.0 | 147.9 | 349.0 | 707.4 | 1454.9 |
| drug-polymer | | | se | 6.6 | 15.4 | 15.2 | 42.0 | 86.1 | 210.6 |
| conjugate IV | | | t/c | 0.97 | 0.58 | 0.46 | 0.60 | 0.64 | 0.69 |
| | | | P value | 0.3752 | 0.0010 | 0.0010 | 0.0278 | 0.0548 | 0.0543 |
| Water-soluble | 0.7 mg/kg | d 0-4 | mean | 137.4 | 144.0 | 120.2 | 300.2 | 547.0 | 926.8 |
| drug-polymer | | | se | 12.6 | 12.8 | 11.5 | 30.6 | 87.8 | 162.0 |
| conjugate III | | | t/c | 1.01 | 0.51 | 0.37 | 0.51 | 0.50 | 0.44 |
| | | | p value | 0.4612 | 0.0002 | 0.0002 | 0.0102 | 0.0151 | 0.0037 |
| compound IV | 0.7 mg/kg | d 0-4 | mean | 137.0 | 155.0 | 132.6 | 276.3 | 529.6 | 1068.8 |
| without water- | | | se | 46 | 52 | 44 | 92 | 177 | 356 |
| soluble portion | | | t/c | 1.01 | 0.55 | 0.41 | 0.47 | 0.48 | 0.50 |
| | | | p value | 0.4655 | 0.0009 | 0.0007 | 0.0077 | 0.0167 | 0.0080 |
| compound III | 0.7 mg/kg | d 0-4 | mean | 136.3 | 192.6 | 198.8 | 489.0 | 745.9 | 1281.7 |
| without water | | | se | 6.2 | 21.8 | 21.2 | 68.8 | 104.9 | 209.8 |
| soluble portion | | | t/c | 1.00 | 0.68 | 0.62 | 0.84 | 0.68 | 0.60 |
| | | | p value | 0.4816 | 0.0104 | 0.0120 | 0.2296 | 0.0792 | 0.0240 |

Cell Culture and Proliferation Assay for Wortmannin Derivatives

A549 (human non-small cell lung cancer) and H-157 cell lines were purchased from American Type Culture Collection (ATCC) (Rockville, Md.). Cells were cultured in RPMI Medium 1640 containing 10% fetal bovine serum (FBS) in a 37° C. incubator containing 5% CO₂. All cell culture reagents were purchased from Gibco-BRL (Grand Island, N.Y.). Cells were plated in 96-well culture plates at about 3000 cells per well. One day following plating, water-soluble drug-polymer conjugates or the vehicle controls were added to cells. Proliferation assays were performed three days post initiation of treatment. For the non-radioactive cell proliferation assay, viable cell densities were determined by measuring metabolic conversion (by viable cells) of the dye MTS tatrazolium dye, a cell proliferation assay known by one of skill in the art (MTS assay), a previously established cell proliferation assay. The assay was performed using assay kit purchased from Promega Corp. (Madison, Wis.). The assay plates were incubated for 1-2 hours and the results were read in a 96-well format plate reader by measuring absorbance at 490 nm. For the thymidine incorporation assay, cells were labeled with [methyl-³H]-thymidine (PerkinElmer Life Sciences, Boston, Mass.) for 5 hours. Cells were then harvested onto glass-fiber filter membranes and counted in a Wallac 1205 Betaplate liquid scintillation counter. Effect of each drug treatment was calculated as a percentage of control cell growth obtained from vehicle-treated cells grown in the same plate.

Effects on In Vitro Proliferation of Human Tumor Cells

| | Thymidine Assay | | MTS Assay | |
|---|---|---|---|---|
| | IC50(μg/mL) | | IC50(μg/mL) | |
| compound | H-157 | A549 | H-157 | A549 |
| Wortmannin | 3.2 | 2.5 | 10.0 | 9.0 |
| Water-soluble | >3 | >3 | >3 | >3 |
| drug-polymer | | | | |
| conjugate IV | | | | |
| Water-soluble | >3 | >3 | >3 | >3 |
| drug-polymer | | | | |
| conjugate III | | | | |
| compound IV | 3.0 | 4.0 | 2.9 | 6.2 |
| without water- | | | | |
| soluble portion | | | | |
| compound III | 9 | 10 | 8 | 10.5 |
| without water- | | | | |
| soluble portion | | | | |

What is claimed is:

1. A water-soluble drug-polymer conjugate having the general formula P—X-D: wherein,
P is a water-soluble polymer;
D is a wortmannin derivative; and
X is a covalent linkage between a water-soluble polymer and the wortmannin derivative.

2. A pharmaceutical composition comprising the water-soluble drug-polymer conjugate of claim 1 and a pharmaceutically acceptable carrier.

3. A method for treating or inhibiting a pathological condition or disorder mediated in a mammal comprising providing to said mammal an effective amount of a water-soluble drug-polymer conjugate of claim 1.

4. A method of claim 3 wherein the effective amount of the water-soluble drug-polymer is 10 to 1000 mg/kg.

5. A method of claim 3 wherein the effective amount of the water-soluble drug-polymer is 0.5 to 10 mg/kg.

6. A method of claim 3 wherein treating or inhibiting comprises inhibition of PI3 kinase.

7. A method of claim 3 wherein treating or inhibiting comprises inhibition of TOR kinase.

8. A method of claim 3 wherein the pathological condition is non-small cell lung cancer.

9. A method of claim 3 wherein the pathological condition is brain cancer, iscaemic heart disease, restenosis, inflammation, platelet aggregation, sclerosis, respiratory disorder, HIV and bone resorption.

10. A method of claim 3 wherein providing an effective amount is alone or in combination with other agents that modulate growth factor signaling, cytokine response, and cell cycle control.

11. A method of claim 10 wherein the agent is interferon-α.

12. A method of claim 10 wherein the agent is pegylated rapamycin.

13. A method of claim 10 wherein the agent is a cytotoxic.

14. A water-soluble drug-polymer conjugate having the structure of formula I 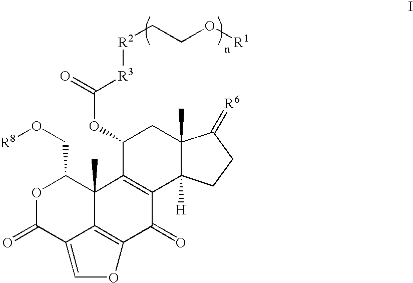
wherein:
R¹ is alkyl, or a drug-polymer conjugate of formula (A) 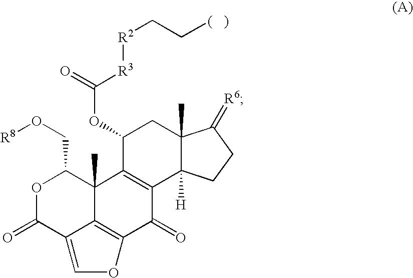
R² is —O—, —NH—, or —S—;
R³ is alkyl, a cycloalkyl, or aryl;
R is ═O or OR⁷;
R⁷ is H, COR⁹ or alkyl;
R⁸ is alkyl or H;
R⁹ is alkyl, H, aryl, or —CH₂Ar; and
n is 1-1000.

15. The water-soluble drug-polymer conjugate of claim 14 wherein n is 250-400.

16. The water-soluble drug-polymer conjugate of claim 14 wherein n is 50-150.

17. The water-soluble drug-polymer conjugate of claim 14 wherein the molecular weight of polymer is from about 400 to about 80,000.

18. The water-soluble drug-polymer conjugate of claim 14 wherein the molecular weight of polymer from about 1000 to about 8000.

19. The water-soluble drug-polymer conjugate of claim 14 wherein the molecular weight of polymer is from about 4000 to about 6000.

20. A pharmaceutical composition comprising the water-soluble drug-polymer conjugate of claim 14 and a pharmaceutically acceptable carrier.

21. A method for treating or inhibiting a pathological condition or disorder mediated in a mammal comprising providing to said mammal an effective amount of a water-soluble drug-polymer conjugate of claim 14.

22. A method of claim 21 wherein the effective amount of the water-soluble drug-polymer is 10 to 1000 mg/kg.

23. A method of claim 21 wherein the effective amount of the water-soluble drug-polymer is 0.5 to 10 mg/kg.

24. A method of claim 21 wherein treating or inhibiting comprises inhibition of P13 kinase.

25. A method of claim 21 wherein treating or inhibiting comprises inhibition of TOR kinase.

26. A method of claim 21 wherein the pathological condition is non-small cell lung cancer.

27. A method of claim 21 wherein the pathological condition is brain cancer, iscaemic heart disease, restenosis, inflammation, platelet aggregation, sclerosis, respiratory disorder, HIV and bone resorption.

28. A method of claim 21 wherein providing an effective amount is alone or in combination with other agents that modulate growth factor signaling, cytokine response, and cell cycle control.

29. A method of claim 28 wherein the agent is interferon-α.

30. A method of claim 28 wherein the agent is pegylated rapamycin.

31. A method of claim 28 wherein the agent is a cytotoxic.

32. A water-soluble drug-polymer conjugate having the structure of formula I: 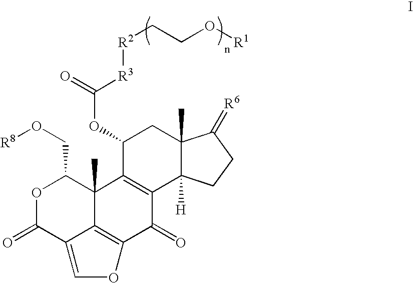
wherein:
R¹ is alkyl, or a drug-polymer conjugate of formula (B) 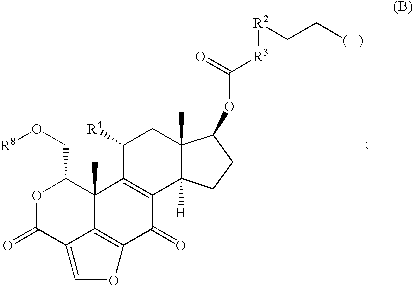
R² is —O—, —NH—, or —S—;
R³ is alkyl, a cycloalkyl, or aryl;
R⁴ is H, ═O, —O—COC₄H₉, or OR⁷;
R⁸ is alkyl or H;
R⁹ is alkyl, H, aryl, or —CH₂Ar; and
n is 1-1000.

33. The water-soluble drug-polymer conjugate of claim 32 wherein n is 250-400.

34. The water-soluble drug-polymer conjugate of claim 32 wherein n is 50-150.

35. The water-soluble drug-polymer conjugate of claim 32 wherein the molecular weight of polymer is from about 400 to about 80,000.

36. The water-soluble drug-polymer conjugate of claim 32 wherein the molecular weight of polymer is from about 1000 to about 8000.

37. The water-soluble drug-polymer conjugate of claim 32 wherein the molecular weight of polymer is from about 4000 to about 6000.

38. A pharmaceutical composition comprising the water-soluble drug-polymer conjugate of claim 32 and a pharmaceutically acceptable carrier.

39. A method for treating or inhibiting a pathological condition or disorder mediated in a mammal comprising providing to said mammal an effective amount of a water-soluble drug-polymer conjugate of claim 32.

40. A method of claim 39 wherein the effective amount of the water-soluble drug-polymer is 10 to 1000 mg/kg.

41. A method of claim 39 wherein the effective amount of the water-soluble drug-polymer is 0.5 to 10 mg/kg.

42. A method of claim 39 wherein treating or inhibiting comprises inhibition of P13 kinase.

43. A method of claim 39 wherein treating or inhibiting comprises inhibition of TOR kinase.

44. A method of claim 39 wherein the pathological condition is non-small cell lung cancer.

45. A method of claim 39 wherein the pathological condition is brain cancer, iscaemic heart disease, restenosis, inflammation, platelet aggregation, sclerosis, respiratory disorder, HIV and bone resorption.

46. A method of claim 39 wherein providing an effective amount is alone or in combination with other agents that modulate growth factor signaling, cytokine response, and cell cycle control.

47. A method of claim 46 wherein the agent is interferon-α.

48. A method of claim 46 wherein the agent is pegylated rapamycin.

49. A method of claim 46 wherein the agent is a cytotoxic.

50. A water-soluble drug-polymer conjugate having the structure of formula II 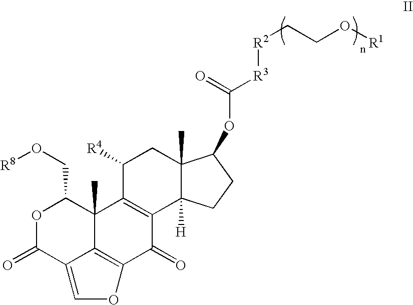
wherein:
R¹ is alkyl, or a drug-polymer conjugate of formula (B) 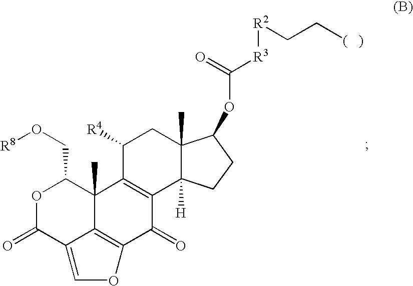
R² is —O—, —NH—, or —S—;
R³ is alkyl, a cycloalkyl, or aryl;
R⁴ is H, ═O, —O—COC₄H₉, or OR⁷;
R⁷ is H, COR⁹ or alkyl;
R⁸ is alkyl or H;
R⁹ is alkyl, H, aryl, or —CH₂Ar; and
n is 1-1000.

51. The water-soluble drug-polymer conjugate of claim 50 wherein n is 250-400.

52. The water-soluble drug-polymer conjugate of claim 50 wherein n is 50-150.

53. The water-soluble drug-polymer conjugate of claim 50 wherein the molecular weight of polymer is from about 400 to about 80,000.

54. The water-soluble drug-polymer conjugate of claim 50 wherein the molecular weight of polymer is from about 1000 to about 8000.

55. The water-soluble drug-polymer conjugate of claim 50 wherein the molecular weight of polymer is from about 4000 to about 6000.

56. A pharmaceutical composition comprising the water-soluble drug-polymer conjugate of claim 50 and a pharmaceutically acceptable carrier.

57. A method for treating or inhibiting a pathological condition or disorder mediated in a mammal comprising providing to said mammal an effective amount of a water-soluble drug-polymer conjugate of claim 50.

58. A method of claim 57 wherein the effective amount of the water-soluble drug-polymer is 10 to 1000 mg/kg.

59. A method of claim 57 wherein the effective amount of the water-soluble drug-polymer is 0.5 to 10 mg/kg.

60. A method of claim 57 wherein treating or inhibiting comprises inhibition of P13 kinase.

61. A method of claim 57 wherein treating or inhibiting comprises inhibition of TOR kinase.

62. A method of claim 57 wherein the pathological condition is non-small cell lung cancer.

63. A method of claim 57 wherein the pathological condition is brain cancer, iscaemic heart disease, restenosis, inflammation, platelet aggregation, sclerosis, respiratory disorder, HIV and bone resorption.

64. A method of claim 57 wherein providing an effective amount is alone or in combination with other agents that modulate growth factor signaling, cytokine response, and cell cycle control.

65. A method of claim 64 wherein the agent is interferon-α.

66. A method of claim 64 wherein the agent is pegylated rapamycin.

67. A method of claim 64 wherein the agent is a cytotoxic.

68. A water-soluble drug-polymer conjugate having the structure of formula III: 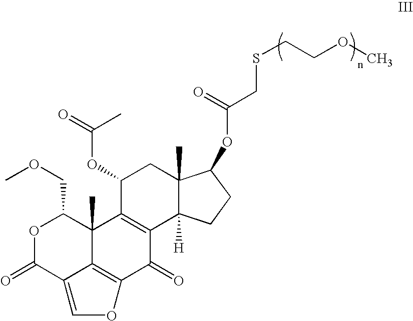
n is 1-1000.

69. The water-soluble drug-polymer conjugate of claim 68 wherein n is 250-400.

70. The water-soluble drug-polymer conjugate of claim 68 wherein n is 50-150.

71. The water-soluble drug-polymer conjugate of claim 68 wherein the molecular weight of polymer is from about 400 to about 80,000.

72. The water-soluble drug-polymer conjugate of claim 68 wherein the molecular weight of polymer is from about 1000 to about 8000.

73. The water-soluble drug-polymer conjugate of claim 68 wherein the molecular weight of polymer is from about 4000 to about 6000.

74. A water-soluble drug-polymer conjugate having the structure of formula IV: 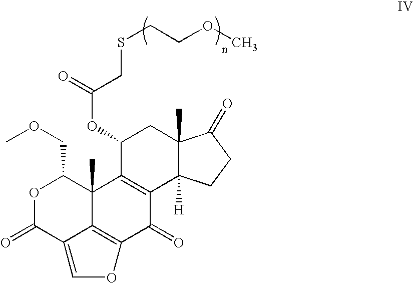
wherein n=1-1000.

75. The water-soluble drug-polymer conjugate of claim 74 wherein n is 250-400.

76. The water-soluble drug-polymer conjugate of claim 74 wherein n is 50-150.

77. The water-soluble drug-polymer conjugate of claim 74 wherein the molecular weight of polymer is from about 400 to about 80,000.

78. The water-soluble drug-polymer conjugate of claim 74 wherein the molecular weight of polymer is from about 1000 to about 8000.

79. The water-soluble drug-polymer conjugate of claim 74 wherein the molecular weight of polymer is from about 4000 to about 6000.

80. A pharmaceutical composition comprising the water-soluble drug-polymer conjugate of claim 74 and a pharmaceutically acceptable carrier.

81. A method for treating or inhibiting a pathological condition or disorder mediated in a mammal comprising providing to said mammal an effective amount of a water-soluble drug-polymer conjugate of claim 74.

82. A method of claim 81 wherein the effective amount of the water-soluble drug-polymer is 10 to 1000 mg/kg.

83. A method of claim 81 wherein the effective amount of the water-soluble drug-polymer is 0.5 to 10 mg/kg.

84. A method of claim 81 wherein treating or inhibiting comprises inhibition of P13 kinase.

85. A method of claim 81 wherein treating or inhibiting comprises inhibition of TOR kinase.

86. A method of claim 81 wherein the pathological condition is non-small cell lung cancer.

87. A method of claim 81 wherein the pathological condition is brain cancer, iscaemic heart disease, restenosis, inflammation, platelet aggregation, sclerosis, respiratory disorder, HIV and bone resorption.

88. A method of claim 81 wherein providing an effective amount is alone or in combination with other agents that modulate growth factor signaling, cytokine response, and cell cycle control.

89. A method of claim 88 wherein the agent is interferon-α.

90. A method of claim 88 wherein the agent is pegylated rapamycin.

91. A method of claim 88 wherein the agent is a cytotoxic.

92. A process for the preparation of a water-soluble drug-polymer conjugate of claim 68 comprising:
a. adding a solvent to 17-dihydro-17-(1-iodoacetyl)-wortmannin to obtain a solution;
b. adding a tertiary amine or sodium bicarbonate to the solution;
C. adding mPEG-sulfhydryl 5000 to the solution of step (b);
d. stirring the solution of step (c) for 30 minutes;
e. adding ether to the stirred solution;
f. collecting the solid; and
g. washing the collected solid with ether to obtain the pegylated wortmannin derivative.

93. A water-soluble drug-polymer conjugate having the structure of formula V: 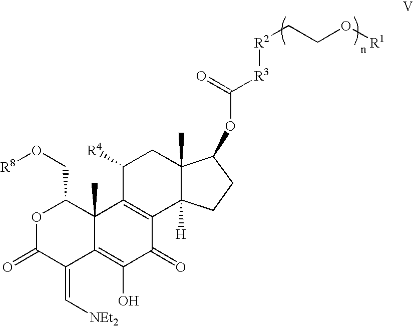
wherein:
R¹ is alkyl, or a drug-polymer conjugate of a single non-repeating formula (V) 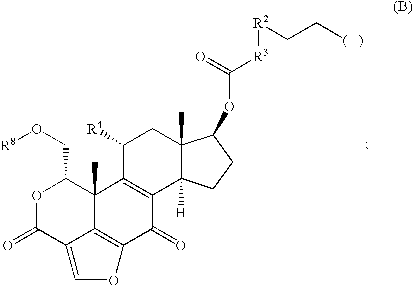
R² is —O—, —NH—, or —S—;
R³ is alkyl, a cycloalkyl, or aryl;
R⁴ is H, ═O, —O—COC₄H₉, or OR⁷;
R⁷ is H, COR⁹ or alkyl;
R⁸ is alkyl or H;
R⁹ is alkyl, H, aryl, or —CH₂Ar; and
n is 1-1000.

94. A process for the preparation of the compound of claim 93 comprising addition of an amine to a compound of claim 50 to obtain a compound of claim 93.

95. A process of claim 94 wherein the amine comprises diethyl amine.

96. A process for the preparation of a water-soluble drug-polymer conjugate of claim 74 comprising:
a) adding a solvent to 11-desacetyl-11-(1-iodoacetyl)-wortmannin to obtain a solution;
b) adding a tertiary amine to the solution;
c) adding mPEG-sulfhydryl 5000 to the solution of step (b);
d) stirring the solution of step (c) for 30 minutes;
e) adding ether to the stirred solution;
f) collecting the solid; and
g) washing the collected solid with ether to obtain the pegylated wortmannin derivative,
as disclosed.
